(19) United States
(12) Patent Application Publication
(10) Pub. No.: US 2004/0258195 A1
(43) Pub. Date: Dec. 23, 2004

(54) Diagnostic X-ray system and CT image production method

(76) Inventor: Yukihiro Hara, Tokyo (JP)

Correspondence Address: PATRICK W. RASCHE, ARMSTRONG TEASDALE LLP, ONE METROPOLITAN SQUARE, SUITE 2600, ST. LOUIS, MO, 63102-2740, US

(21) Appl. No.: 10/869,381

(22) Filed: Jun. 16, 2004

(30) Foreign Application Priority Data
Jun. 17, 2003 (JP) 2003-172526

(51) Int. Cl. G21K 1/12; A61B 6/00; G01N 23/00

(52) U.S. Cl. 378/11

(57) ABSTRACT

A diagnostic X-ray system has an X-ray source that generates an X-ray conical beam, and a panel detector, which has numerous X-ray detection elements arrayed two-dimensionally, opposed to each other so that they can be rotated about the body axis of a subject. CT images of the subject are reconstructed based on projection data acquired by scanning the subject. The panel detector can be offset so that the center of the detecting surface of the panel detector extending in a direction perpendicular to the body axis can be displaced in a direction substantially perpendicular to a reference plane defined with an extension of a straight line linking the focal point of X-rays and a point on the body axis of the subject and with the body axis of the subject.

### BACKGROUND OF THE INVENTION

The present invention relates to a diagnostic X-ray system and a CT image production method. More particularly, the present invention relates to a diagnostic X-ray system that has an X-ray source which generates an X-ray conical beam, and a panel detector, which has numerous X-ray detection elements arrayed two-dimensionally, opposed to each other so that the X-ray source and panel detector can be rotated about the body axis of a subject, and that reconstructs computed tomography (CT) images of the subject on the basis of projection data acquired by scanning the subject. The present invention also relates to a method of producing such CT images.

In recent years, a technique (angiographic CT) of concurrently performing diagnosis and therapy on a subject using a combination of an X-ray CT system and an angiography system has been adopted (Patent Document 1). In an angiographic CT system, an angiography system is first used to produce images of a vascular region characterized with a contrast medium. Immediately afterward, an X-ray CT system installed in the same room as the angiography system is used to produce CT images of the vascular region. The angiographic CT system is therefore clinically highly evaluated.

Moreover, in recent years, an attempt has been made to adapt an X-ray flat-panel detector (FPD) to diagnostic X-ray systems (angiography system and X-ray CT system) for the purpose of performing fluoroscopy or computed tomography on a subject. For example, a conventionally known X-ray fluoroscopic imaging system has first and second shields positioned anteriorly and posteriorly to a subject in order to limit an X-ray field, and uses the FPD to detect fluoroscopic images of a subject so as to align the X-ray field with a desired region of the subject (Patent Document 2).

[Patent Document 1]

Japanese Unexamined Patent Publication No. 2003-102717 (abstract and drawings)

[Patent Document 2]

Japanese Unexamined Patent Publication No. 2003-061941 (abstract and drawings)

However, although large FPDs exist, the size is about 40 cm by 40 cm. If such an FPD is employed, a field of view (hereinafter FOV) within which a subject is observed while being scanned to produce CT images and which is determined with an FPD is about 20 cm in diameter. The FPD is too small to be adopted for computed tomography (CT) of a human body.

### SUMMARY OF THE INVENTION

Therefore, an object of the present invention is to provide a diagnostic X-ray system capable of performing high-definition CT on subjects ranging from a relatively small-size subject to a relatively large-size subject despite a compact and simple configuration, and a CT image production method.

The foregoing problems are solved with the configuration shown in, for example, FIG. 1. Namely, a diagnostic X-ray system in accordance with the aspect (1) of the present invention has an X-ray source 31 which generates an X-ray conical beam, and a panel detector 42, which has numerous X-ray detection elements arrayed two-dimensionally, opposed to each other so that they can be rotated about the body axis CLb of a subject A. The diagnostic X-ray system reconstructs CT images of the subject on the basis of projection data acquired by scanning the subject A. The panel detector 42 can be offset so that the center 42c of the detecting surface of the panel detector 42 extending in a direction perpendicular to the body axis CLb can be displaced in a direction substantially perpendicular to a reference plane defined with an extension of a straight line linking a focal point F of X-rays and a point on the body axis CLb of a subject and with the body axis CLb of the subject.

Referring to FIG. 1, when the relatively large-size subject A is scanned by performing CT, the FPD 42 is slid in, for example, a direction of arrow b. The focal point F of X-rays, the body axis CLb of the subject A, and the left edge of the detecting surface of the FPD 42 are aligned with one another in a y-axis direction in the drawing. In this state, the X-ray imaging assembly is rotated about the body axis CLb.

An inlet (a) shows a view (0) of projection data acquired with the X-ray source set at a view angle 0°. Since the FPD 42 is offset or displaced rightwards in the drawing, projection data is acquired from the right half of the subject A in the drawing. Likewise, an inlet (b) shows a view (90) of projection data acquired with the X-ray source set at a view angle 90°. At this time, projection data is acquired from the upper half of the subject A in the drawing. An inlet (c) shows a view (180) of projection data acquired with the X-ray source set at a view angle 1800. At this time, projection data is acquired from the left half of the subject A in the drawing.

The foregoing data acquisition will be discussed in relation to the whole of the subject A. When the X-ray imaging assembly is rotated from 0° to 180°, all projection data items required to reconstruct an image of the whole of the subject A observed from the position of the view angle 0° are gathered. When the X-ray imaging assembly is further rotated by 180° (that is, from 180° to 360°), projection data items required to reconstruct images of the whole of the subject A and usually acquired by performing a half scan are gathered. Consequently, CT images of the subject A can be reconstructed according to the known back projection technique or the like.

According to the aspect (1) of the present invention, CT is performed with the FPD 42 held off-centered. CT of a relatively large-size subject can be achieved readily for a short period of time with a little load imposed on the subject. Moreover, numerous projection data items can be acquired from the direction of the body axis of a subject during one scan. Consequently, three-dimensional CT or volume CT can be readily performed on the relatively large-size subject.

According to the aspect (2) of the present invention, the detecting surface of the panel detector 42 employed according to the aspect (1) of the present invention has, as shown in, for example, FIG. 6(c), a flat surface. The panel detector can be manufactured readily.

According to the aspect (3) of the present invention, the panel detector 42 employed according to the aspect (2) of the present invention is, as shown in, for example, FIG. 9, offset so that an X-ray CLx passing through the center 42c of the detecting surface of the panel detector and the detecting surface thereof will meet at right angles. In this case, fluoroscopic images of a subject are projected around the center of the detecting surface of the detector. Consequently, image reconstruction can be achieved readily, and CT image quality is improved.

According to the aspect (4) of the present invention, the detecting surface of the panel detector 42 employed according to the aspect (1) of the present invention is, as shown in, for example, FIG. 11 (d), part of a cylindrical surface that extends in the direction of the body axis CLb with the focal point F of X-rays as a center thereof. Consequently, the same image reconstruction as the one adopted for a multi-detector employed in conventional X-ray CT systems can be utilized, and CT image quality is improved.

According to the aspect (5) of the present invention, the detecting surface of the panel detector 42 employed according to the aspect (1) of the present invention is, as shown in, for example, FIG. 11 (e), part of a spherical surface having the focal point F of X-rays as a center thereof. Consequently, an X-ray field can be homogenized in the direction of the body axis of a subject, and CT image quality is improved.

According to the aspect (6) of the present invention, the diagnostic X-ray system in accordance with the aspect (1) of the present invention further comprises: a designating means that is used to designate a first radiographic mode in which a relatively small-size subject is radiographed, or a second radiographic mode in which a relatively large-size subject is radiographed; and a panel control means that when the first radiographic mode is designated, aligns the center of the detecting surface of the panel detector with an extension of a straight line linking the focal point of X-rays and a point on the body axis of a subject (see FIG. 6), and that when the second radiographic mode is designated, offsets the panel detector so that the center of the detecting surface of the panel detector will be displaced in a direction substantially perpendicular to a reference plane defined with the extension of the straight line and the body axis of the subject (see FIG. 7). Consequently, despite the compact configuration and simple control, high-definition CT images of subjects ranging from a relatively small-size subject to a relatively large-size subject can be produced readily.

According to the aspect (7) of the present invention, when the second radiographic mode is designated, the panel control means employed according to the aspect (6) of the present invention offsets the panel detector 42 so that the edge of the detecting surface of the panel detector 42 will, as shown in, for example, FIG. 7, substantially cross the extension of the straight line linking the focal point F of X-rays and a point on the body axis CLb of a subject. Consequently, the finite detecting surface of the panel can be utilized at the maximum.

According to the aspect (8) of the present invention, the focal point F of X-rays described in relation to the aspect (1), (2), (4), or (5) of the present invention can be, as shown in, for example, FIG. 10, displaced in the same direction as the panel detector 42 can. According to the aspect (8) of the present invention, the focal point F of X-rays is displaced in the same direction as the panel detector 42 is. Consequently, a large-size subject can be radiographed under the same radiographic conditions as those under which a small-size subject is radiographed.

According to the aspect (9) of the present invention, the diagnostic X-ray system in accordance with the aspect (8) of the present invention further comprises, as shown in, for example, FIG. 10: a designating means that is used to designate a first radiographic mode in which a relatively small-size subject is radiographed, or a second radiographic mode in which a relatively large-size subject is radiographed; and an imaging assembly control means that when the first radiographic mode is designated, aligns the focal point of X-rays and the center of the detecting surface of the panel detector with a predetermined straight line perpendicular to the body axis of the subject, and that when the second radiographic mode is designated, displaces the focal point of X-rays and the center of the detecting surface of the panel detector in the direction substantially perpendicular to the reference plane defined with the predetermined straight line and the body axis of the subject. Consequently, despite the compact configuration and simple control, high-definition CT images of subjects ranging from a relatively small-size subject to a relatively large-size subject can be produced readily.

According to the aspect (10) of the present invention, when the second radiographic mode is designated, the imaging assembly control means employed according to the aspect (9) of the present invention, as shown in, for example, FIG. 10, offsets the panel detector so that the edge of the detecting surface of the panel detector will substantially cross the predetermined straight line. Consequently, the finite detecting surface of the panel can be utilized at the maximum.

According to the aspect (11) of the present invention, the center of the detecting surface of the panel detector described in relation to the aspect (1), (2), (4), or (5) of the present invention can be displaced in the direction of the straight line linking the focal point of X-rays and a point on the body axis of a subject. Even when the panel detector is lifted or lowered in the direction of the focal point of X-rays, an FOV within which a subject is observed can be varied.

According to the aspect (12) of the present invention, the diagnostic X-ray system in accordance with the aspect (1) or (8) of the present invention further comprises: as shown in FIG. 2 and FIG. 3, a C-arm 23 that bears the X-ray source 31 and panel detector 42 so that they can be rotated about the body axis CLb of a subject; and a display means on the monitor screen 63 of which fluoroscopic images of the subject A detected by the panel detector 42 can be displayed in real time. The system configuration for CT in accordance with the present invention is preferably adapted to angiography systems (X-ray TV systems). Unlike related arts, an angiography system and a CT system need not be installed separately. This leads to a great reduction in an installation space and a cost.

According to the aspect (13) of the present invention, the X-ray imaging assembly employed according to the aspect (1) or (8) of the present invention includes an X-ray source 71 and a panel detector 75 and is incorporated in a gantry 76 that can be rotated about the body axis of a subject. The system configuration for CT in accordance with the present invention is preferably adapted to X-ray CT systems.

A CT image production method in accordance with the aspect (14) of the present invention is adapted to a diagnostic X-ray system set forth in claim 1, and comprises: a step of scanning a subject with a panel detector offset; a step of synthesizing projection data items, which are acquired with the X-ray source set at mutually opposite first and second view angles during the scan, to adopt the synthetic projection data as projection data to be acquired from the whole of the subject with the X-ray source set at the first or second view angle; a step of producing the synthetic projection data relative to all view angles within at least 180°; and a step of reconstructing CT images of the subject on the basis of the produced projection data items.

A CT image production method in accordance with the aspect (15) of the present invention is adapted to a diagnostic X-ray system set forth in claim 8, and comprises: a step of scanning a subject with a focal point of X-rays displaced and with a panel detector offset; a step of synthesizing projection data items, which are acquired with an X-ray source set at mutually opposite first and second view angles during the scan, to adopt the synthetic projection data as projection data to be acquired from the whole of a subject with the X-ray source set at the first or second view angle; a step of producing the synthetic projection data relative to all view angles within at least 180°, and a step of reconstructing CT images of the subject on the basis of the produced projection data items.

A program in accordance with the aspect (16) of the present invention can be run in a computer in order to direct the computer to execute the CT image production method set forth in claim 14 or 15.

According to the present invention, high-definition CT can be readily performed on any subject ranging from a relatively small-size subject to a relatively large-size subject despite the compact and simple configuration. This greatly contributes to expansion of the application range of diagnostic X-ray systems and improvement in performance thereof.

Further objects and advantages of the present invention will be apparent from the following description of the preferred embodiments of the invention as illustrated in the accompanying drawings.

### BRIEF DESCRIPTION OF THE DRAWINGS

FIG. 1 is an explanatory diagram concerning the principles of the present invention.

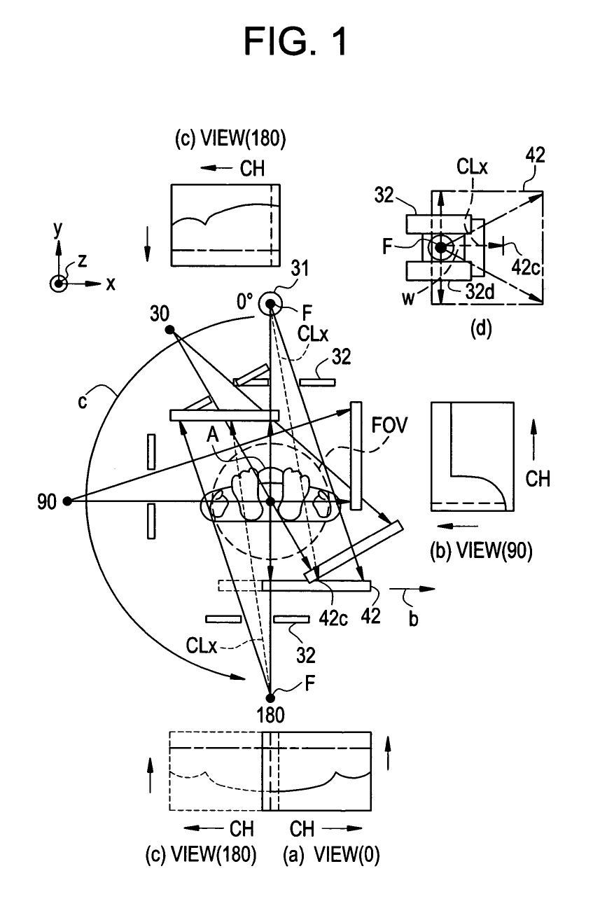

FIG. 2 is a diagram (1) showing the configuration of an angiographic CT system in accordance with an embodiment.

FIG. 3 is a diagram (2) showing the configuration of the angiographic CT system in accordance with the embodiment.

FIG. 4 is an explanatory diagram (1) showing a flat-panel detector employed in the embodiment.

FIG. 5 is an explanatory diagram (2) showing the flat-panel detector employed in the embodiment.

FIG. 6 is an explanatory diagram (1) concerning movements to be made during CT according to the embodiment.

FIG. 7 is an explanatory diagram (2) concerning movements to be made during CT according to the embodiment.

FIG. 8 shows the configuration of an X-ray CT system in accordance with the present embodiment.

FIG. 9 is an explanatory diagram (1) concerning a CT method in accordance with other embodiment.

FIG. 10 is an explanatory diagram (2) concerning a CT method in accordance with other embodiment.

FIG. 11 is an explanatory diagram (3) concerning a CT method in accordance with other embodiment.

### SUMMARY OF THE INVENTION

A preferred embodiment of the present invention will be described in conjunction with appended drawings below. For all the drawings, the same reference numerals denote the same or equivalent components.

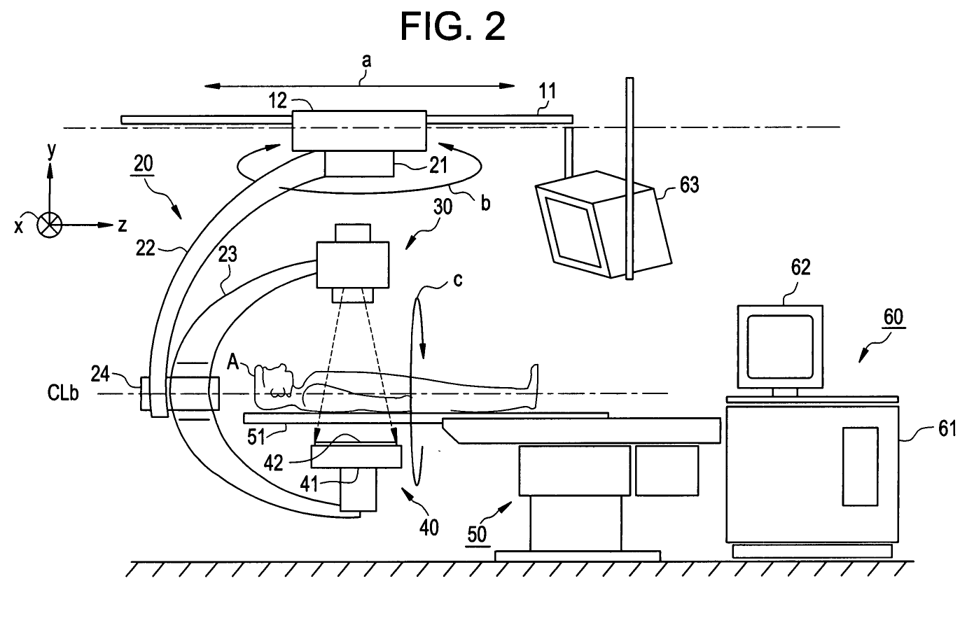

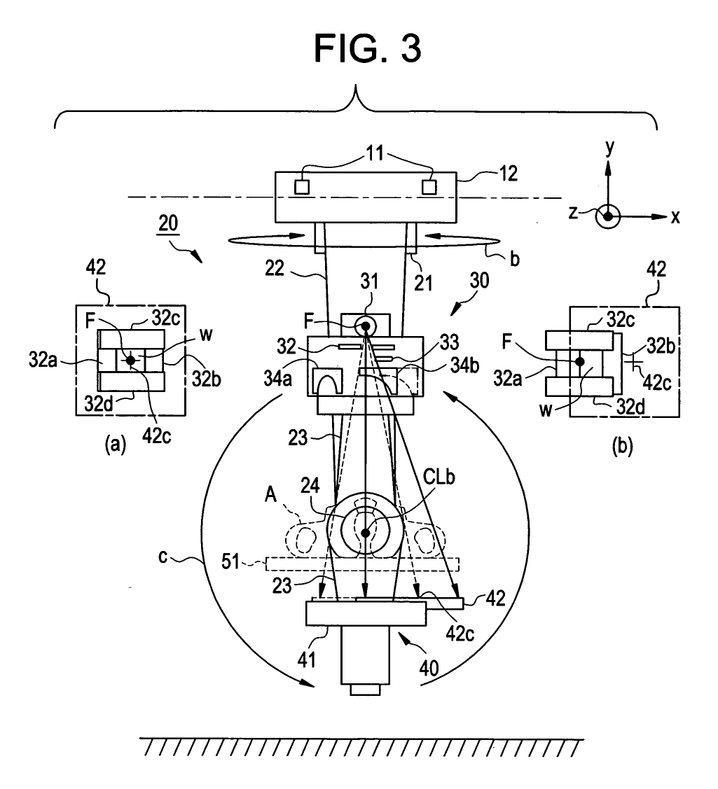

FIG. 2 and FIG. 3 are diagrams (1) and (2) showing the configuration of an angiographic CT system in accordance with an embodiment. Herein, one system has the capabilities of both an angiography system and an X-ray CT system. FIG. 2 is a side view. The system comprises broadly: an X-ray imaging assembly 20 that produces fluoroscopic images of a subject A; a radiographic table 50 on which the subject A lies down and which moves in the directions of the body axis CLb; a TV monitor 63 via which the fluoroscopic images of the subject A can be monitored; and an operating console 60 which a co-medical or the like operates to control the components.

Two shafts (rails) 11 are embedded in a z-axis direction in the ceiling. A support base 12 that bears the X-ray imaging assembly 20 is mounted on the shafts 11 so that the X-ray imaging assembly 20 can be moved in the directions of arrow a along the shafts 11. A support arm 22 is developed below the support base 12. A C-arm 23 that supports the X-ray imaging assembly 20 pivots on the lower end of the support arm 22. The support arm 22 can be swiveled in the directions of arrow b by means of a driving mechanism 21 mounted on the ceiling. Consequently, the X-ray imaging assembly 20 can be approached to the subject A from any direction. However, when the subject A is radiographed by performing CT, the support arm 22 is, as illustrated, positioned to meet the body axis CLb of the subject. Moreover, the C-arm 23 can be rotated in the direction of arrow C by means of a rotating mechanism 24. Consequently, not only fluoroscopic imaging of the subject A but also CT thereof can be performed. An X-ray generator 30 is mounted on one end of the C-arm 23. An imager 40 that produces fluoroscopic images or CT images of the subject A is mounted on the other end of the C-arm 23 so that it will be opposed to the X-ray generator 30. The imager 40 comprises an FPD 42, and a supporting board 41 that supports the FPD 42 so that the FPD 42 can slide in an x axis-direction in the drawing.

FIG. 3 is a front view showing the X-ray imaging assembly 20 from the direction of the body axis of a subject. The X-ray generator 30 comprises: a rotating anode X-ray tube 31 that generates an X-ray conical beam; a collimator 32 that defines an X-ray field; a reference-channel detector 33 that corrects a drift in an exposure; and a bow-tie filter 34 capable of expanding a dynamic range for projection data by improving the uniformity (linearity) in a transmitted dose reaching the FPD 42 according to the sectional form of a subject. On the other hand, the imager 40 includes the FPD 42 and the supporting board 41 that supports the FPD 42 so that the FPD 42 can be offset to be displaced rightwards (or leftwards) in the drawing.

Inlets (a) and (b) are plan views of the collimator 32. One example of the collimator 32 has four X-ray shields 32a to 32d assembled rectangularly. At least the right and left shields 32b and 32a are slid with a focal point F of X-rays fixed, whereby CT can be performed with a subject observed within a relatively small FOV and a relatively large FOV.

The inlet (a) shows the position of the collimator at the time of performing CT while observing a subject within a small FOV. When CT is performed with a subject observed within the small FOV, the focal point F of X-rays, a point on the body axis CLb of a subject, and the center 42 of the detecting surface of the FPD 42 are aligned with one another in a vertical direction in the drawing. Moreover, the opening w of the collimator 32 is adjusted so that the entire detecting surface of the FPD 42 will fall within an X-ray field.

The inlet (b) shows the position of the collimator at the time of performing CT while observing a subject within a large FOV. When CT is performed with a subject observed within the large FOV, the focal point. F of X-rays, a point on the body axis CLb of a subject, and the left edge of the detecting surface of the FPD 42 are aligned with one another in the vertical direction in the drawing. Moreover, the opening w of the collimator 32 is adjusted so that the entire detecting surface of the FPD will fall within an X-ray field.

The bow-tie filter 34 is made of Teflon® or the like. The X-ray generator 30 includes a filter 34a for use in performing CT while observing a subject within a small FOV, and a filter 34b for use in performing CT while observing a subject within a large FOV. The filter 34a is shaped substantially like a horseshoe and elongated in the direction of the body axis. When CT is performed with a subject observed within the small FOV, the opening w shown in the inlet (a) is blocked using the entire filter 34a. On the other hand, the filter 34b is shaped like a bisection of a horseshoe, which is large in an x-axis direction in the drawing, with respect to a straight line extending in the direction of the body axis CLb. When CT is performed with a subject observed within a large FOV, the opening w shown in the inlet (b) is blocked using the entire filter 34b.

When fluoroscopy or fluoroscopic imaging is performed on a subject, the filters 34a and 34b are removed from the opening w in order to attain the same level of image quality (contrast) as the image quality of conventional radiographic images.

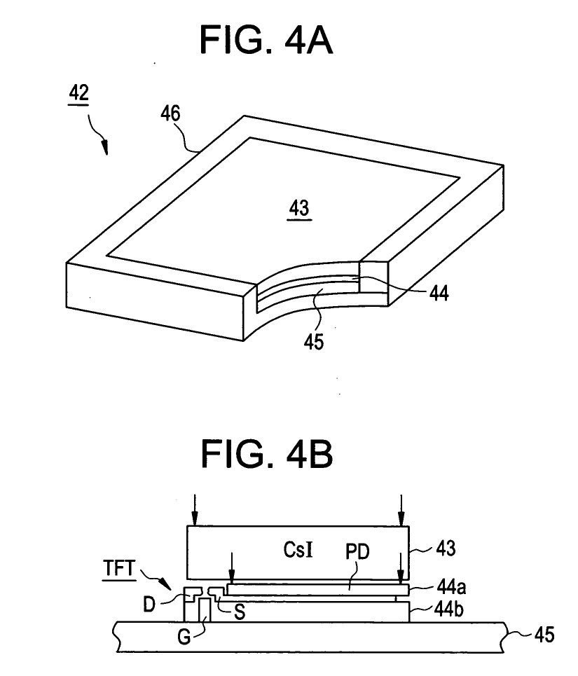

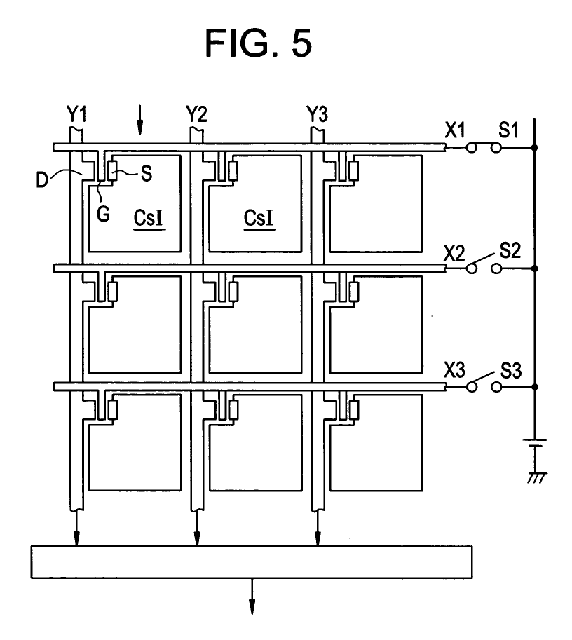

FIG. 4 and FIG. 5 are explanatory diagrams (1) and (2) showing a flat-panel detector (FPD) employed in the present embodiment. FIG. 4(A) is a perspective view (cutaway) showing the appearance of the FPD 42. In FIG. 4(A), reference numeral 43 denotes a scintillator layer serving as a detecting surface that converts X-rays into light (X-ray photons). Reference numeral 44 denotes an amorphous silicon layer that converts the light into charge and reads the charge. Reference numeral 45 denotes a substrate layer made of a glass or the like. Reference numeral 46 denotes an outer case that bears and accommodates the layers. The external size of an example of the FPD 42 is 50 cm in length by 50 cm in width. An effective area in which X-rays can be received is calculated as 41 cm in length by 41 cm in width. A receptor is divided into numerous pixel locations. A pitch between adjoining pixel locations is expressed as 200 μ by 200 μ. An image matrix includes 2022 pixels in rows and 2022 pixels in columns, and has about 4.9 million pixels in total.

FIG. 4(B) is a side sectional view of one pixel location. The scintillator layer 43 contains cesium iodide (Cs1) as a fluorescent substance. Owing to the needle-like special columnar crystalline structure of cesium iodide, dispersion of X-ray photons within the scintillator that causes a blur in a radiographic image is limited. In the amorphous silicon layer 44, a photodiode layer 44a that converts light, which is produced by the scintillator layer 43, into charge, and a field effect transistor (FET) switching array layer 44b that reads the resultant charge from each pixel location are formed by utilizing a thin film transistor (TFT) technology.

FIG. 5 is a plan view showing part of the FPD 42 in enlargement. A voltage is applied to the gate G of a switching FET, and charge resulting from photoelectric conversion and being stored in the photodiode PD included in each pixel location is read as a current. Namely, a voltage is applied to a scan line X1, and charges stored in line are read in Y directions (read lines) at a time. Thereafter, a voltage is applied to a scan line X2, and charges stored in line are read in the Y directions at a time. The same applies to the subsequent scan lines. The thus read currents are converted into voltages, and then analog-to-digital converted. Consequently, radiographic (projection) data containing 4 bits per pixel is produced.

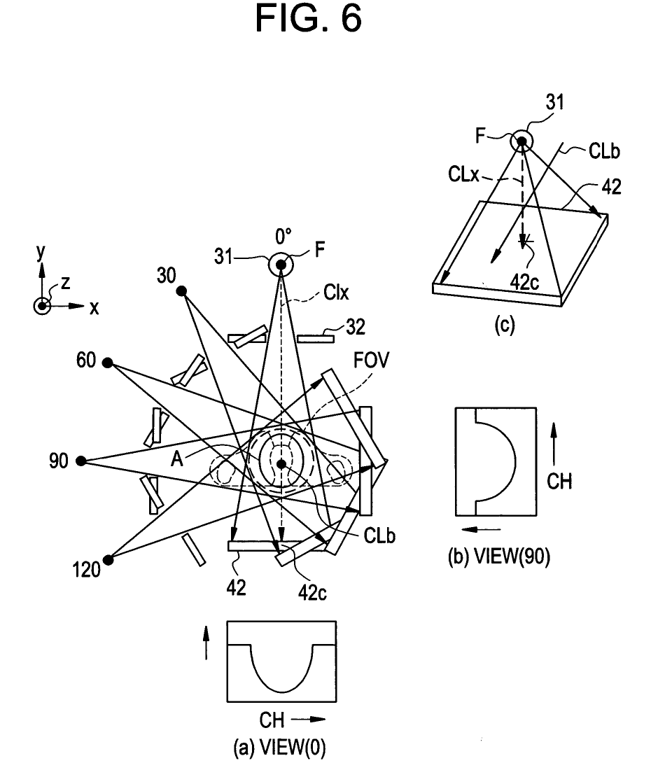

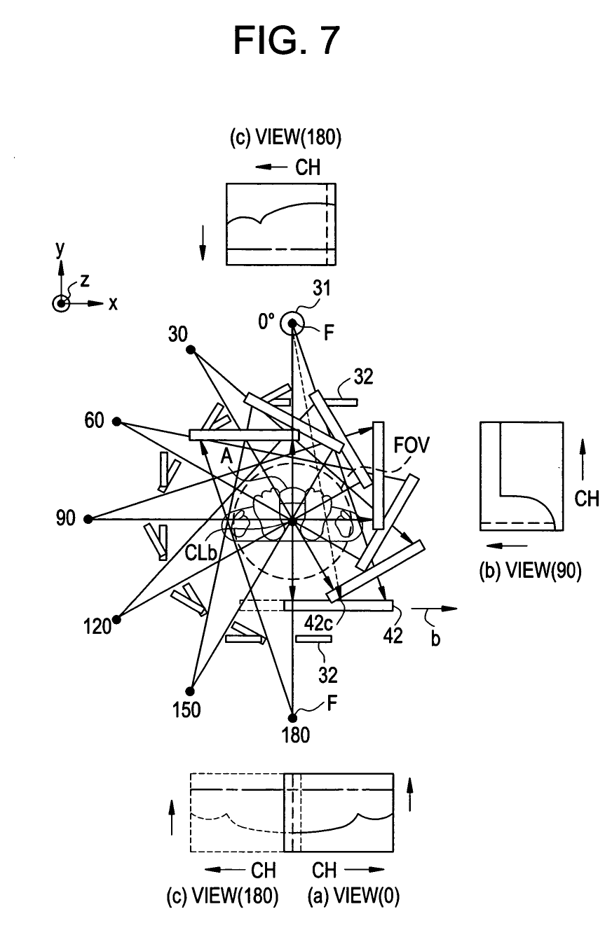

FIG. 6 and FIG. 7 are explanatory diagrams (1) and (2) indicating movements to be made during CT according to the present embodiment. Incidentally, the radiographic movements to be made by an angiography system (X-ray TV system) can be inferred from Patent Document 1, and the description of the movements will therefore be omitted. Moreover, an inlet (c) is a perspective view showing the appearance of the FPD 42 employed in the present embodiment. The detecting surface of the panel is a flat surface.

FIG. 6 shows movements to be made during CT that is performed with a subject observed within a relatively small FOV (about 20 cm in diameter). The CT to be performed with a subject observed within the small FOV is adapted to high-definition CT to be performed on, for example, small animals or a human head. Referring to FIG. 6, when CT is performed with a subject observed within the relatively small FOV, the focal point F of X-rays, a point on the body axis CLb of the subject A, and the center 42c of the detecting surface of the FPD 42 are aligned with one another in a y-axis direction in the drawing (vertical direction). In this state, the C-arm 23 is rotated about the body axis CLb.

An inlet (a) indicates a view (0) of projection data acquired with the X-ray tube set at a view angle 0°. The axis of abscissas indicates channels lined in an x-axis direction in the drawing, and the axis of ordinates indicates amplitudes of projection data. As seen from the indications of amplitudes, small doses are detected on channels on which X-rays transmitted by the subject A fall. Likewise, an inlet (b) indicates a view (90) of projection data acquired with the X-ray tube set at a view angle 90°. When CT is performed with a subject observed within a small FOV, the whole of the subject A falls within an X-ray field needed to acquire each view. The C-arm 23 is rotated at least 180 (half scan), whereby CT images of the subject A can be reconstructed according to the known back projection technique adopted for general X-ray CT systems. Needless to say, the C-arm 23 may be rotated 360° (full scan). According to the present embodiment, when the FPD 42 having numerous detection elements arrayed even in a body-axis direction is employed, numerous projection data items can be acquired simultaneously even in the direction of the body axis of a subject during one scan. Consequently, so-called three-dimensional CT or volume CT can be readily realized.

FIG. 7 shows movements to be made during CT that is performed with a subject observed within a relatively large FOV (40 cm or more in diameter). The CT to be performed with a subject observed within the large FOV can be adapted to high-definition CT to be performed on a human trunk. When CT is performed with a subject observed within the relatively large FOV, the FPD 42 is slid in, for example, the direction of arrow b. Thus, the focal point F of X-rays, a point on the body axis CLb of the subject A, and the left edge of the detecting surface of the FPD 42 are aligned with one another in a y-axis direction in the drawing. In this state, the C-arm 23 is rotated about the body axis CLb.

An inlet (a) indicates a view (0) of projection data acquired with the X-ray tube set at a view angle 0°. Since the FDP 42 is offset to be displayed rightwards in the drawing, projection data is acquired from the right half of the subject A in the drawing. Likewise, an inlet (b) indicates a view (90) of projection data acquired with the X-ray tube set at a view angle 90°. At this time, projection data is acquired from the upper half of the subject A in the drawing. Furthermore, an inlet (c) indicates a view (180) of projection data acquired with the X-ray tube set at a view angle 180°. At this time, projection data is acquired from the left half of the subject A.

The above data acquisition will be discussed in relation to the whole of the subject A. When the C-arm 23 is rotated from 0° to 180°, all projection data items required to reconstruct an image of the whole of the subject A observed from the position of the view angle 0° are gathered. Therefore, the C-arm 23 is further rotated at least 180° (namely from 180° to 360°), whereby projection data items that are usually acquired by performing a half scan and required to reconstruct the images of the whole of the subject A are gathered. The CT images of the whole of the subject A are reconstructed according to the known back projection technique. Needless to say, the C-arm 23 may be rotated 360° or more (full scan).

Incidentally, all projection data items needed to reconstruct the image of the subject observed from the view angle 0° include the view (0) of projection data acquired with the X-ray tube set at the view angle 0° and the view (180) of projection data acquired with the X-ray tube set at the view angle 180°. The projection data items are synthesized (joined). If the projection data items cannot be joined because of missing data, a defect such as a ring artifact is likely to occur in images. Therefore, preferably, projection data items acquired at positions that are mutually opposite at an angle of 180° are used to interpolate intermediate projection data. Images are then reconstructed using the projection data items.

FIG. 7 shows an example of a mode in which a subject is scanned while observed within a large FOV. The present invention is not limited to this mode. For example, the subject A and FPD 42 may be lifted in the y-axis direction in the drawing, and the opening of the collimator 32 may be widened at the same time. Thus, CT can be readily performed with a subject observed within a larger FOV.

Moreover, an FOV varies depending on an offset by which the FPD 42 is displaced in the direction of arrow b. According to the present embodiment, CT can be performed with high definition while a subject is observed within an FOV of any size.

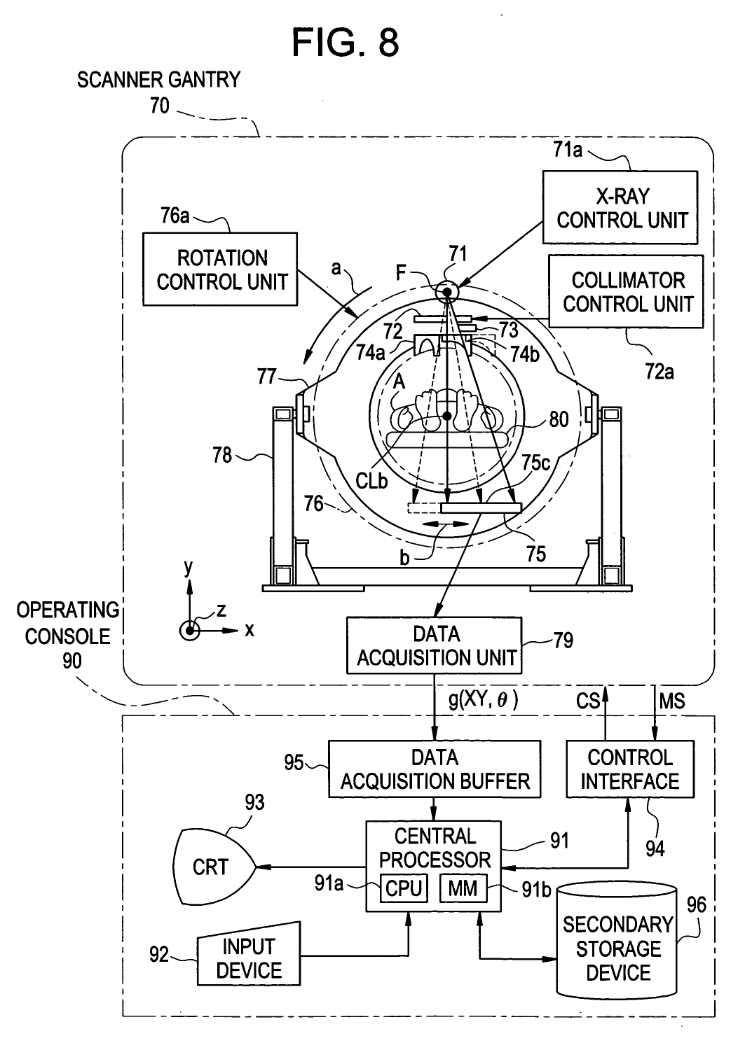

FIG. 8 shows the configuration of an X-ray CT system in accordance with the present embodiment, thus showing an example of an X-ray CT system to which a CT method in accordance with the present invention is adapted. The X-ray CT system comprises broadly: a scanner gantry 70 that scans the subject A with an X-ray conical beam; a radiographic table 80 on which the subject A lies down and which is moved in the directions of the body axis CLb; and an operating console 90 which a co-medical or the like operates to control the components 70 and 80.

In the scanner gantry 70, reference numeral 71 denotes a rotating anode X-ray tube, and reference numeral 71 a denotes an X-ray control unit that controls an X-irradiation time and an intensity of X-rays (tube voltage kV, tube current mA). Reference numeral 72 denotes a collimator that defines an X-ray field, and reference numeral 72a denotes a collimator control unit that controls the X-ray field. Reference numeral 73 denotes a reference-channel detector that corrects a drift in an exposure. Reference numeral 74 denotes a bow-tie filter used to expand a dynamic range for projection data. Reference numeral 75 denotes a flat-panel detector (FPD) capable of sliding in the direction of arrow b in the drawing. Reference numeral 76 denotes a gantry that holds the X-ray imaging assembly including the components 71 to 75 so that the X-ray imaging assembly can be rotated about the body axis CLb of a subject. Reference numeral 76a denotes a gantry rotation control unit. Reference numeral 77 denotes a back plate that bears the entire gantry 76 so that the gantry can be rotated. Reference numeral 78 denotes a support frame that supports a support arm 77 so that the support arm 77 can be tilted. Reference numeral 79 denotes a data acquisition unit that produces or acquires projection data from the subject A on the basis of signals detected by the FPD 75.

In the operating console 90, reference numeral 91 denotes a central processor that is responsible for major control and processing to be extended or performed in the X-ray CT system (scan control and reconstruction of CT images). Reference numeral 91a denotes a CPU incorporated in the central processor 91. Reference numeral 91b denotes a main memory (MM) formed with a RAM or ROM and used by the CPU 91a. Reference numeral 92 denotes an input device that is used to enter commands or data and that includes a keyboard and a mouse. Reference numeral 93 denotes a display device (CRT) on which scan schedule information or CT images are displayed. Reference numeral 94 denotes a control interface via which various control signals CS and a monitor signal MS are transferred among the CPU 91a, scanner gantry 70, and radiographic table 80. Reference numeral 95 denotes a data acquisition buffer in which projection data acquired by the data acquisition unit 79 is temporarily stored. Reference numeral 96 denotes a secondary storage device (hard disk drive or the like) in which projection data sent from the data acquisition buffer 95 is stored or saved, and in which various application programs and various operation/correction data files that are needed to operate the X-ray CT system are saved.

Actions to be performed for X-ray CT will be described below. A conical beam generated from the X-ray tube 71 is transmitted by the subject A and incident on the FPD 75. The data acquisition unit 79 produces projection data items g(XY,θ) that are associated with outputs of the FPD 75. The projection data items are stored in the data acquisition buffer 95. Herein, XY denotes a channel number assigned to each of the channels in the FPD 75 (that is, matrix numbers), and θ denotes a view angle at which the gantry 76 is set. Furthermore, after the gantry 76 is rotated and set at each view angle θ, X-ray projection is performed. Thus, projection data items are acquired and stored during one rotation of the gantry 76. If necessary, the radiographic table (tabletop) 80 may be intermittently or continuously moved according to the axial/helical scanning technique. Thus, all projection data items acquired from a given scan field of the subject A may be stored. After a scan is fully completed, or while a scan is under way, the CPU 91a reconstructs the CT images of the subject A on the basis of acquired projection data items, and displays them on the display device 93.

According to the present embodiment, when the FPD 75 is held centered or off-centered, high-definition CT that is performed with a subject observed within any FOV ranging from a relatively small FOV (about 20 cm in diameter) to a relatively large FOV (40 cm or more in diameter) can be achieved readily. When CT is performed with a subject observed within the relatively small FOV, the focal point F of X-rays, a point on the body axis CLb of the subject A, and the center 75c of the detecting surface of the FPD 75 are aligned with one another in a y-axis direction in the drawing. In this state, the gantry 76 is rotated about the body axis CLb. Moreover, when CT is performed with a subject observed within the large FOV, the FPD 75 is slid, for example, rightwards in the drawing. Consequently, the focal point F of X-rays, a point on the body axis CLb of the subject A, and the left edge of the detecting surface of the FPD 75 are aligned with one another in the y-axis direction in the drawing. In this state, the gantry 76 is rotated about the body axis CLb. According to the present embodiment, even when the X-ray CT system employs the FPD 75, high-definition and large-volume CT images can be readily produced by acquiring projection data with a subject observed within an FOV of any size ranging from a relatively small FOV to a relatively large FOV.

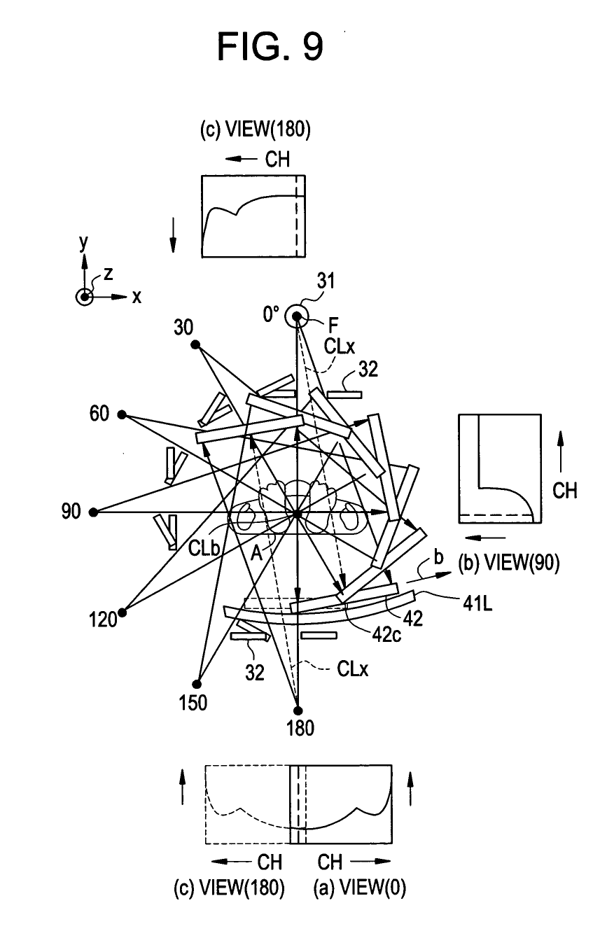

FIG. 9 to FIG. 11 are explanatory diagrams (1) to (3) concerning a CT method in accordance with other embodiments, showing some other X-ray imaging assemblies that are different from one another in the structures of components. Hereinafter, a description will be made on the assumption that the X-ray imaging assemblies are adapted to the angiographic CT system shown in FIG. 2 and FIG. 3. Needless to say, the X-ray imaging assemblies may be adapted to the X-ray CT system shown in FIG. 8.

FIG. 9 shows a case where the FPD 42 is slid so that the central X-ray CLx traveling from the focal point F of X-rays to the center 42c of the detecting surface of the FPD 42 will always fall on the detecting surface at right angles. In order to meet this condition, an arc-shaped guide rail 41L that is shaped like an arc of a circle whose center lies at the position of the focal point F of X-rays is formed in an x-axis direction in the drawing. The FPD 42 is slid in the direction of arrow b along the guide rail 41L, whereby the above condition is met. According to the present embodiment, since the detecting surface of the FPD 42 faces the focal point F all the time, the entire detecting surface exhibits a flat detection characteristic (resolution). Consequently, a distortion or an artifact in CT images can be greatly alleviated.

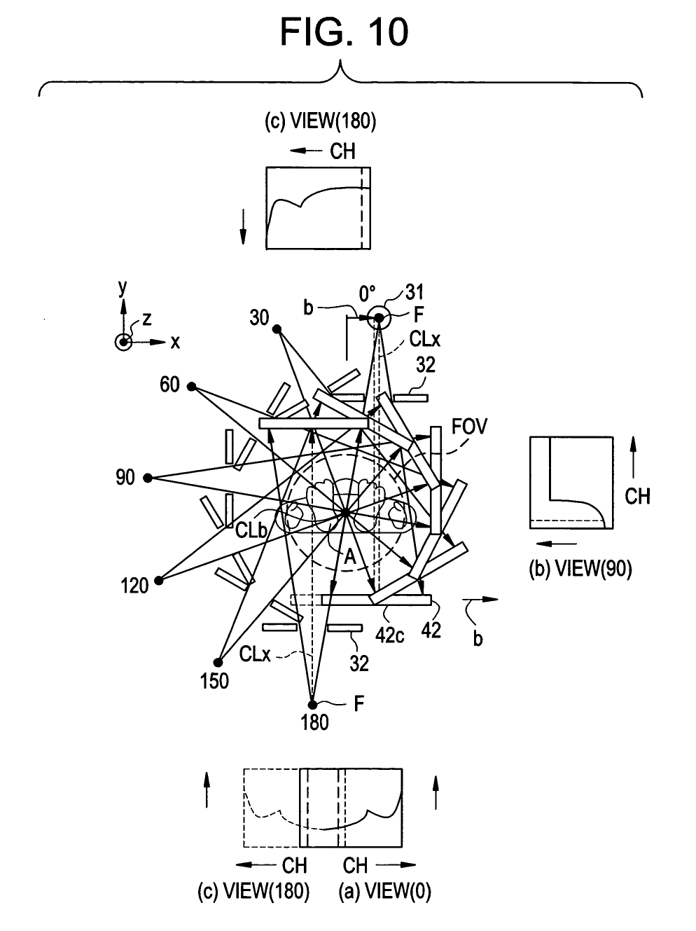

FIG. 10 is concerned with a case where CT is performed with a subject observed within a large FOV and not only the FPD 42 but also the focal point F of X-rays are concurrently slid in the direction of arrow b. Consequently, the same relationship as that shown in FIG. 9 is established, and CT image quality is improved.

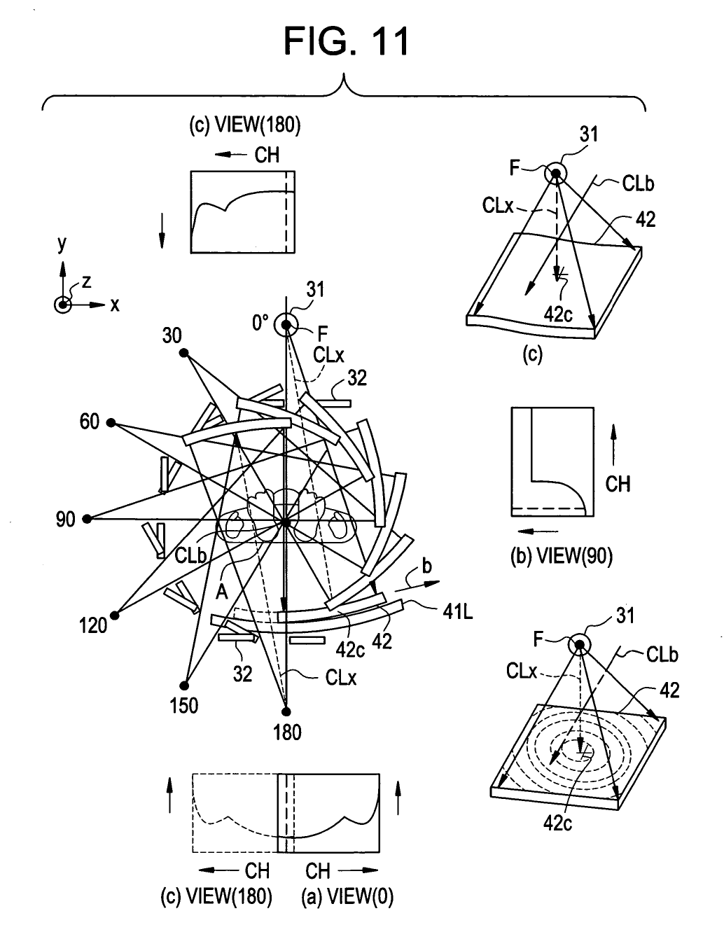

FIG. 11 is concerned with a case where the detecting surface of the FPD 42 is formed as a spherical surface that is part of a sphere whose center lies at the position of the focal point F of X-rays or a cylindrical surface extending parallel to the direction of the body axis CLb. An inlet (d) is a perspective view showing the appearance of the FPD 42 whose detecting surface is formed as a cylindrical surface. When the detecting surface of the FPD 42 is formed as a cylindrical surface, the same image processing method as the one employed in X-ray CT systems having a conventional multi-detector can be adopted. High-definition and high-quality CT images can be reconstructed.

An inlet (e) is a perspective view showing the appearance of the FPD 42 whose detecting surface is formed as a spherical surface. When the detecting surface of the FPD 42 is formed as a spherical surface, a more flat detection characteristic (resolution) can be attained not only in an x-axis direction in the drawing but also in a z-axis (body-axis) direction therein. Consequently, high image quality is ensured.

In the foregoing embodiments, the panel detector 42 is offset so that the center 42c of the detecting surface of the panel detector 42 will be displaced from its reference position. The present invention is not limited to this mode. Alternatively, any point on a line extending from the center of the detecting surface of the panel detector 42 in a direction perpendicular to the body axis CLb can be adopted as the reference position.

The plurality of preferred embodiments of the present invention has been described so far. The configurations of the units or assemblies, a way of controlling the units or assemblies, processing to be performed in the units or assemblies, or a combination thereof may be modified in various manners without a departure from the idea of the present invention.

1. A diagnostic X-ray system that has an X-ray source which generates an X-ray conical beam, and a panel detector, which has numerous X-ray detection elements arrayed two-dimensionally, opposed to each other so that they can be rotated about the body axis of a subject, and that reconstructs CT images of the subject on the basis of projection data acquired by scanning the subject, wherein:
the panel detector can be offset so that the center of the detecting surface of the panel detector extending in a direction perpendicular to the body axis will be displaced in a direction substantially perpendicular to a reference plane defined with an extension of a straight line linking a focal point of X-rays and a point on the body axis of the subject and with the body axis of the subject.

2. A diagnostic X-ray system according to claim 1, wherein the detecting surface of the panel detector is a flat surface.

3. A diagnostic X-ray system according to claim 2, wherein the panel detector is offset so that an X-ray passing through the center of the detecting surface of the panel detector and the detecting surface thereof will meet at right angles.

4. A diagnostic X-ray system according to claim 1, wherein the detecting surface of the panel detector is part of the surface of a cylinder that extends in the direction of the body axis with the focal point of X-rays as a center thereof.

5. A diagnostic X-ray system according to claim 1, wherein the detecting surface of the panel detector is part of the surface of a sphere having the focal point of X-rays as a center thereof.

6. A diagnostic X-ray system according to claim 1, comprising: a designating device for use in designating a first radiographic mode in which a relatively small-size subject is radiographed, or a second radiographic mode in which a relatively large-size subject is radiographed; and a panel control device that when the first radiographic mode is designated, aligns the center of the detecting surface of the panel detector with the extension of the straight line linking the focal point of X-rays and a point on the body axis of a subject, and that when the second radiographic mode is designated, offsets the panel detector so that the center of the detecting surface of the panel detector will be displaced in the direction substantially perpendicular to the reference plane defined with the extension of the straight line and the body axis of a subject.

7. A diagnostic X-ray system according to claim 6, wherein when the second radiographic mode is designated, the panel control device offsets the panel detector so that the edge of the detecting surface of the panel detector will substantially cross the extension of the straight line linking the focal point of X-rays and a point on the body axis of the subject.

8. A diagnostic X-ray system according to claim 1, wherein the focal point of X-rays can be displaced in the same direction as the panel detector can.

9. A diagnostic X-ray system according to claim 8, comprising: a designating device for use in designating a first radiographic mode in which a relatively small-size subject is radiographed, or a second radiographic mode in which a relatively large-size subject is radiographed; and an imaging assembly control device that when the first radiographic mode is designated, aligns the center of the detecting surface of the panel detector with a predetermined straight line perpendicular to the body axis of a subject, and that when the second radiographic mode is designated, displaces the focal point of X-rays and the center of the detecting surface of the panel detector in a direction substantially perpendicular to a reference plane defined with the predetermined straight line and the body axis of a subject.

10. A diagnostic X-ray system according to claim 9, wherein when the second radiographic mode is designated, the imaging assembly control device offsets the panel detector so that the edge of the detecting surface of the panel detector will substantially cross the predetermined straight line.

11. A diagnostic X-ray system according to claim 1, wherein the panel detector can be offset so that the center of the detecting surface of the panel detector will be displaced in the direction of the straight line linking the focal point of X-rays and a point on the body axis of a subject.

12. A diagnostic X-ray system according to claim 1, further comprising: a C-arm that supports an X-ray source and the panel detector so that they can be rotated about the body axis of a subject; and a display device on the monitor screen of which fluoroscopic images of the subject detected by the panel detector are displayed in real time.

13. A diagnostic X-ray system according to claim 1, wherein an X-ray imaging assembly including the X-ray source and panel detector is incorporated in a gantry that can be rotated about the body axis of a subject.

14. A CT image production method, comprising the steps of: scanning a subject with the panel detector included in the diagnostic X-ray system of claim 1 offset; synthesizing projection data items, which are acquired with the X-ray source set at first and second view angles during the scan, so as to adopt the synthetic projection data as projection data acquired from the whole of the subject with the X-ray source set at the first or second view angle; producing the synthetic projection data relative to all view angles within at least 180°; and reconstructing CT images of the subject on the basis of the produced projection data items.

15. A CT image production method, comprising the steps of: scanning a subject with the focal point of X-rays and panel detector, which are employed in the diagnostic X-ray system of claim 8, displaced; synthesizing projection data items, which are acquired with the X-ray source set at mutually-opposite first and second view angles during the scan, so as to adopt the synthetic projection data as projection data acquired from the whole of the subject with the X-ray source set at the first or second view angle; producing the synthetic projection data relative to all view angles within at least 180°; and reconstructing CT images of the subject on the basis of the produced projection data items.
